(19) United States
(12) Patent Application Publication
(10) Pub. No.: US 2011/0033817 A1
(43) Pub. Date: Feb. 10, 2011

(54) MINIMALLY INVASIVE INSTRUMENT AND METHOD TO TREAT PERIODONTAL DISEASE

(75) Inventor: William F. McKay, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/909,673

(22) Filed: Oct. 21, 2010

Related U.S. Application Data
(63) Continuation of application No. 12/423,396, filed on Apr. 14, 2009.
Application No. 12/423,396 is a continuation of application No. PCT/US2010/030846, filed on Apr. 13, 2010.

(51) Int. Cl. A61C 17/26; A61B 1/24; A61C 17/28; A61C 17/00

(52) U.S. Cl. 433/29; 433/131; 433/91; 433/82

(57) ABSTRACT

The invention provides an instrument and methods for minimally invasive surgical scaling and/or planning procedures, thus reducing patient recovery time. Furthermore, the present invention overcomes the shortcoming in the prior art of inadequately preparing the diseased site or creating an aerosol by providing a device that is more mechanically abrasive by way of utilizing a rotational mechanical action of a plurality of bristles.

### CROSS-REFERENCE TO RELATED APPLICATIONS

This application is a continuation of U.S. patent application Ser. No. 12/423,396, filed Apr. 14, 2009, and International Patent Application No. PCT/US2010/030846, filed Apr. 13, 2010, the entirety of each of which are incorporated by reference.

### BACKGROUND

This application relates generally to devices and methods for removal of diseased periodontal tissue, more particularly, to devices and methods for the removal of plaque, calculus, tartar, stain, inflamed lose soft connected tissue, top layer of adjacent bone surfaces, and/or other substances associated with periodontal disease.

Any publications or references discussed herein are presented to describe the background of the invention and to provide additional detail regarding its practice. Nothing herein is to be construed as an admission that the inventors are not entitled to antedate such disclosure by virtue of prior invention.

Currently dentist and periodontist treat moderate and severe periodontal disease surgically. Gingival flap surgery or a variation thereof is typically used to gain access to the diseased bone. This procedure requires that the gums be separated from the teeth and folded out of the way to allow the dentist to access the root of the tooth and/or the bone supporting the teeth. Diseased, inflamed, or otherwise unwanted tissue is removed from area around the teeth and any defects in the supporting jaw bone. Once the tissue is removed, plaque and calculus are removed by scaling and root planning. Upon completion of the surgery, the gums are placed back against the teeth and secured in place.

In mild periodontal disease, the treatment typically consists solely of non-surgical scaling and root planning. Scaling and root planning, or non-surgical periodontal therapy, is a method of removing dental plaque, calculus; tartar, stain, cementum, and/or surface dentin which are thought to cause inflammation, from the root of a tooth, thereby helping to reestablish a healthy periodontal environment. The procedure is currently conducted with either ultrasonic instruments and/or hand instruments, for example, periodontal scalers and curettes. However, many ultrasonic scalers do not adequately remove diseased tissue or prepare adjacent bone surfaces for subsequent application or regenerative therapy. They also require some form of irrigation or liquid output to cool the tool while it is being used. In addition, ultrasonic scalers vibrate at such a high frequency, to remove the plaque and other material, that they create aerosols that can then spread pathogens to other areas that were previously uninfected.

Currently there are no effective minimally invasive therapies for the treatment of moderate to severe periodontal disease. The invention overcomes this problem by providing an instrument and method of adequately preparing the diseased site and treating periodontal disease using a minimally invasive approach. The invention also provides a device that can be used without producing an aerosol and spreading pathogens.

### SUMMARY OF THE INVENTION

The invention provides an instrument and method that can prepare a tooth root surface, adjacent bone, and/or surrounding soft tissue with results similar to or better than the present standard for a surgical scaling and planning procedure. In addition, the present invention provides an instrument and method that is minimally invasive, thus reducing patient recovery time. Furthermore, the present invention overcomes the shortcoming in the prior art of inadequately preparing the diseased site or creating an aerosol by providing a device that is more mechanically abrasive, or does not induce the formation of an aerosol.

The periodontal device of the invention has the ability to loosen and/or remove any diseased or necrosed tissue in a periodontal disease pocket adjacent to a tooth with a rotating mechanical action.

The invention relates to a periodontal device comprising a motor, a drive shaft rotatably connected to the motor, a plurality of bristles detachably connected to the drive shaft and a power supply casing substantially covering at least the motor and drive shaft, wherein the device is configured such that the motor supplies rotational energy through the drive shaft to the plurality of bristles, and wherein the plurality of bristles have a maximal diameter of travel during rotation of less than about 8 mm, less than about 7 mm, less than about 6 mm, less than about 5 mm, less than about 4 mm, less than about 3 mm, less than about 2 mm, or less than about 1 mm. Optionally, the drive shaft is flexible and/or steerable, thereby allowing the operator to maneuver the bristles in a precise way in a very confined space.

The invention also relates to a periodontal device where a first mating fitting is connected to the drive shaft and a second mating fitting rotatably connects the plurality of bristles, such that rotational energy from the drive shaft can be transmitted to rotational motion of the bristles. In an exemplary embodiment, the plurality of bristles may have sufficient rigidity to resist deflection due to centripetal forces associated with their rotation during operation of the device. The plurality of bristles may also have sufficient rigidity to break-up diseased connective soft tissue and the top layer of adjacent bone surfaces to prepare a site for regenerative therapy application. The periodontal device further comprising a fluid irrigation and/or a suction feature(s) for removing loose tissue.

Further, the invention may employ a first sheath, wherein the plurality of bristles may be retracted within the first sheath, and, optionally, the first sheath may cover at least a part of the drive shaft up to the entire drive shaft.

The invention also relates to a periodontal device coupled with an illuminating system configured to illuminate a region around the rotatable bristles, and an imaging system positioned along the length of the drive shaft such that the illuminating system illuminates tissue near the bristles and the imaging system transmits visual images of the tissue proximal to the bristles to a monitor.

The invention also relates to a periodontal device comprising a plurality of rotatable bristles where the bristles may have a smooth surface, a rough surface, a rounded distal tip, a shaped distal tip, and/or a grinding or polishing material adhered to at least the distal tip.

The invention also relates to a kit comprising the motor, drive shaft and a housing, along with a plurality of tips having different sizes or shapes.

The invention also relates to a method of removing undesirable tissue or detritus from around a tooth, the method comprising inserting a periodontal device comprising a plurality of bristles, rotating the bristles in contact with the undesirable tissue and removing the undesirable tissue or detritus, for example, by fluid irrigation and/or a suction.

### BRIEF DESCRIPTION OF THE FIGURES

The accompanying drawings, which are hereby incorporated into and constitute a part of this specification, illustrate various embodiments of the invention and, together with the description, serve to explain the principles of the invention. In the drawings like reference numerals represent like parts:

FIG. 1 is a frontal view of an exemplary embodiment.

FIG. 2 is a cross-sectional view of an exemplary embodiment.

FIG. 3 A is a cross-sectional view of a detachable distal end of the tip that is substantially the same size as the extension in FIG. 2. FIG. 3 B is a cross-sectional view of a detachable distal end of the tip that is substantially larger than the expansion in FIG. 2. FIG. 3 C is a cross-sectional view of a detachable distal end of the tip wherein the bristles have a whisk-like configuration.

FIG. 4 is an expanded view of the area labeled F of FIG. 3B, illustrating an exemplary embodiment, wherein an additional length of the bristles is held on a spool and releasably extendable trough a holding member and a clutch system that have an open position wherein the bristle can extend past the holding member to extend their length in the device and a closed position wherein the bristles are held firmly at a fixed length.

FIG. 5 is an expanded view of the distal end of the tip, wherein FIG. 5A shows a shallow bend and FIG. 5B shows an approximately 90 degree bend.

FIG. 6 illustrates an exemplary embodiment having a steering means, a illumination means and a viewing means, along with the bristles in the tip.

FIG. 7A is a partial cross-sectional view of an exemplary embodiment wherein the bristles are retracted within the tip. FIG. 7b is a partial cross-sectional view of an exemplary embodiment wherein the bristles are extended beyond the tip.

FIG. 8 is a partial cross-sectional view of an exemplary embodiment wherein an extendable sheath 90 forms a retractable cannula around the tip 4.

FIG. 9 illustrates various bristle forms. FIG. 9A is a partial cross-sectional view of bristle having a smooth outer surface and a rounded tip. FIG. 9B is a partial cross-sectional view of bristle having a rough outer surface and a rounded tip. FIG. 9C is a partial cross-sectional view of bristle having a shaped tip. FIG. 9d is a partial cross-sectional view of bristle having a grinding or polishing material applied to the tip.

### DETAILED DESCRIPTION OF THE INVENTION

For the purposes of promoting an understanding of the principles of the invention, reference will now be made to certain embodiments and specific language will be used to describe the same. Nevertheless, it will be understood that no limitation of the scope of the invention is intended by the reference to the embodiments and that alterations and further modifications of the illustrated device, along with further applications of the principles of the invention described herein, will be recognized in light of the present disclosure by one skilled in the art to which the invention relates.

The uses of the terms “a” and “an” and “the” and similar references in the context of describing the invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, each separate value in the range is incorporated into the specification as if it were individually recited herein.

All methods described herein may be performed in any suitable order unless otherwise indicated or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “comprising,” “including,” “containing,” “characterized by,” and grammatical equivalents thereof are inclusive or open-ended terms that do not exclude additional, unrecited elements or method steps, but will also be understood to include the more restrictive terms “consisting of” and “consisting essentially of.”

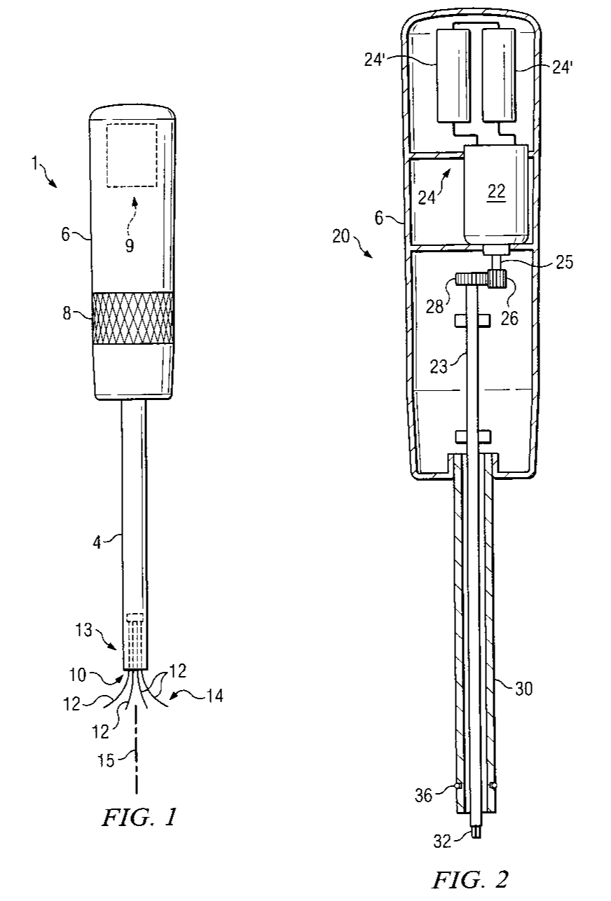

Referring to FIG. 1, the invention provides a device 1 comprising a tip portion 4, a power supply body or handle 6 (which may optionally further comprise a gripping area 8 (a grip)), and a power supply 9, where optionally all or part of the tip portion 4 may have interchangeable inserts that allow the geometry or size of the tip 4 to be changed. Extending from an opening 10 in the distal end of the tip 4 are a plurality of bristles 12, each having a proximal end 13 connected to a drive shaft (not shown) within the tip, and a distal end 14 where centripetal force due to rotation of the bristles 12 extends the distal end 14 of the bristle 12 away from a center axis running along line 15-15. Alternatively, the bristles 12 may have a pre-formed shape that remains substantially unchanged in response to centripetal forces produced by rotation of the bristle 12 around the central axis 15-15.

It will be appreciated that the illustrated shape of the power supply body or handle 6, grip 8 and tip 4 are merely exemplary and that the handle 6, grip 8 and tip 4 can be formed to have any desirable shape. Preferably, the shapes of the handle 6, grip 8 and tip 4 are such that the device 1 is ergonomically efficient and pleasant to handle by the user.

Referring to FIG. 2, the device 20 may comprise a source of rotational force, such as an electrical, pressurized air turbine or other known motor 22, which directly or indirectly connects with a drive shaft 23 located within the tip 4. For example, the motor 22 may be an electrical motor 24 (e.g., a DC motor 24 having a battery source 24′) that may be connected indirectly to the drive shaft 23 by way of a motor shaft 25 terminating in a motor gear 26 designed to engage a gear 28 that is connected to the drive shaft 23. In this configuration a gear box may be inserted between the motor 22 and the drive shaft 23 allowing for the control of rotational speed of the bristles 12 by way of selecting an appropriate gearing ratio. Alternatively, the motor 22 may be directly connected to the drive shaft 23. The rotation speed of the drive shaft may be controlled and/or varied according to any known methods or devices, including the use of motors 22 and circuits described in U.S. Pat. No. 6,329,783, and the like

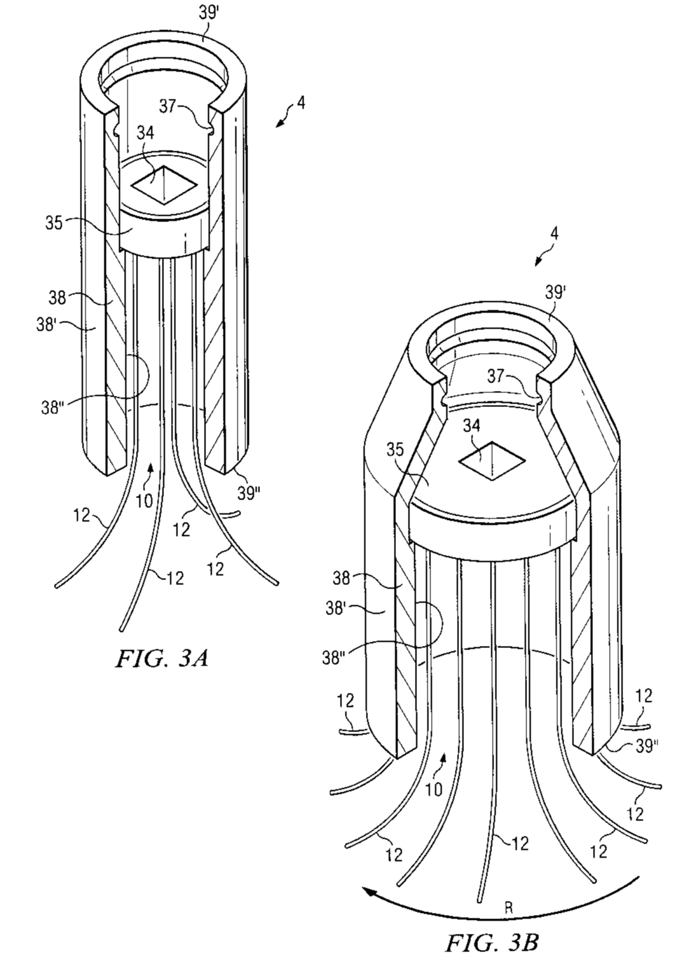

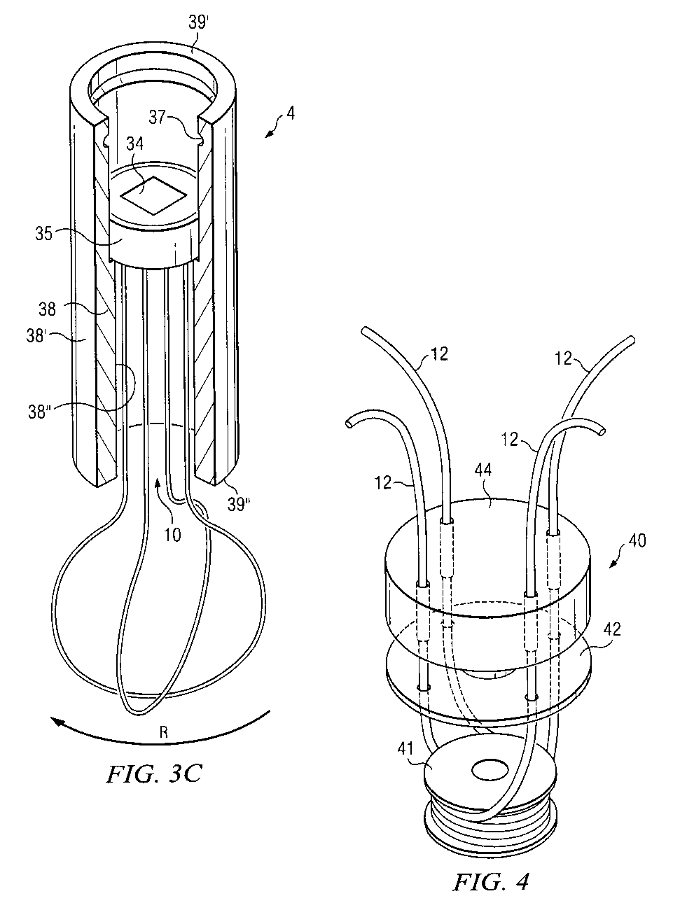

The tip 4 may comprise a unitary elongate member connected to the power supply body or handle 6, as illustrated in FIG. 1. Alternatively, the tip 4 may be connected to the motor indirectly, for example the tip 4 as illustrated in FIGS. 3A-C may be snap fitted onto extension 30 illustrated in FIG. 2.

In these embodiments utilizing a detachable tip 4 (FIG. 3A), the drive shaft 23 will have to be detachably coupled to the rotatably mounted bristles 12 (FIGS. 2 and 3A-C). For example, the drive shaft 23 may terminate at or in a first mating fitting 32 that is adapted to mate with a second mating fitting 34. In FIGS. 2 and 3A-C the first mating fitting 32 is illustrated as a relatively square block 32 connected to the drive shaft 23 and the second mating fitting 34 is illustrated as a relatively square shaped socket 34, wherein the socket 34 is adapted to receive the block 32 and thereby couple the rotational force applied to the drive shaft 23 to the bristles 12. As will be recognized, the mating fittings 32, 34 may be of any shape or size. The second mating fitting 34 may be contained in a rotational member 35 which directly or indirectly connects with the bristles 12, and secures rotation of the bristles 12 in a plane substantially parallel with the plane defined by the opening 10 in the distal end of the tip 4.

The tip 4 may be adapted to detachably connect to an extension 30 and the motor 22 through any mechanism know in the art, for example, by way of a frictional snap fitting using a ridge 36 on the outer surface of extension 30 and groove 37 on the inner surface of the tip 4.

The tips 4 in FIGS. 3A-C comprise a cylindrical wall 38 having an outside surface 38′ and an inside surface 38″. In FIG. 3A the diameter of the inside surface 38″ of the cylindrical wall 38 remains relatively constant from the top of the cylinder 39′ to the bottom of the cylinder 39″. The top of the cylinder 39′ is, as discussed herein, configured to detachably connect to the extension 30 and the drive shaft 23 such that the bristles 12 can be rotated in the direction “R.” In FIG. 3B the top 39′ of the cylindrical wall 38 is still sized to receive the extension 30, however, the bottom of the cylinder 39″ has a larger diameter than the top of the cylinder 39′, thereby providing a tip 4 with a larger working area. Likewise, the bottom diameter 39″ may be smaller than the top of the cylinder 39′, thereby providing a tip 4 with a smaller working area. Thus, the invention provides a kit comprising a set of tips having different sized working areas or bristles 12 made of different material. As will now be understood in light of the present disclosure, the working area of the tip will be defined by the outermost path during rotation of the distal end of the bristles 12. In an exemplary embodiment, the tip(s) 4 have a working area of less than about 8 mm, less than about 7 mm, less than about 6 mm, less than about 5 mm, less than about 4 mm, less than about 3 mm, less than about 2 mm or less than about 1 mm.

The bristles 12 may be made of almost any material, such as stainless steel, nickel alloys like Nitinol or plastics. The primary consideration in selecting the material will be its impact resistance, brittleness, hardness and elasticity. For example, if the device 1 is to be used for roughening the surface of the adjacent bone, the material needs to be hard enough to cut into bone. In contrast, if the device 1 is to be used to remove only necrosed soft tissue and some plaque, the material should be relatively soft and have a high elasticity.

In an exemplary embodiment, the device may be configured using an induction motor 22 and a variable frequency controller (not shown) to allow the user to adjust the rotational speed of the bristles 12, for example, the device 1 may operate at low speed, medium speed or high speed, alternatively the rotational speed my be set at any speed between zero revolutions per minute to the maximum number of revolutions per minute for the particular motor 22 and any associated gears 26, 28.

While FIGS. 2-3 illustrate a detachable fitting having a ridge 36 and groove 37 connection, in light of the present specification it will now be apparent that the connection between the tip 4 and extension 30 may be of any size or structure. For example, the tip 4 and extension 30 may be detachably coupled using any of the known ferrule systems, threaded coupling, snap fit or other known attachment systems. The detachability of the tip 4 allows for the use of multiple differentially sized tips 4, bristles 12 made from different materials or tips 4 and/or bristles 12 having different patterns and/or properties, which provides the ability to incrementally remove damaged tissue, plaque and calculus as desired.

In FIG. 4 the bristles 12 are illustrated seated in a clutch type devise 40, where a clutch plate 42 can be disengaged to allow the bristles 12 to extend up though a retaining member 44 and further out the distal end 10 of the tip 4. Rotational force exerted on the bristles 12 can be used to provide the force necessary for extension past the clutch plate 44 and up through the retaining member 44. Thus, the bristles 12 may be held in place and selectively released for extension using any known line feed system (see U.S. Pat. No. 4,136,446).

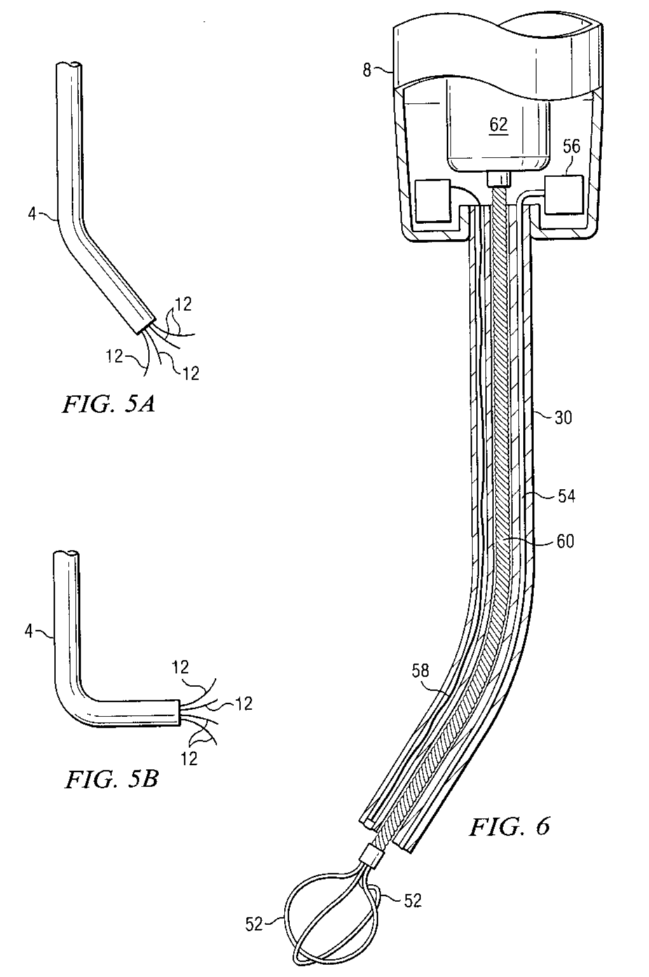

FIG. 5 illustrates that the tip 4 may be shaped according to the applicable use. For example, the tip 4 may have a moderate bend angle (FIG. 5A) between 1 and 85 degrees, for example less than about 10°, 15°, 20°, 25°, 30°, 35°, 40°, 45°, 50°, 55°, 60°, 65°, 70°, 75°, 80°, or 85°, or the tip 4 may have a sharp bend angle of about 90°, for example between about 95° and 85°.

In FIG. 6, the present invention provides a device comprising a plurality of rotatable bristles 52, in combination with an illuminating system or means 54, such as light-emitting diodes or fiber optic light guides connected to a light source 56 so as to illuminate the region around the rotatable bristles 52, and an imaging system or means 58, such as a miniature video camera or a fiber optic image guide. These components are positioned along the length of the rotatable shaft 60 that connects the rotatable bristles 52 with a motor 62, wherein the rotatable shaft 60, illuminating system or means 54 and the imaging system or means 58 comprise a unified flexible member that may be steered through tissue in the oral-maxillofacial area. Examples of illuminating systems, imaging systems and steerable tips may be found in U.S. Pat. No. 7,479,106 and U.S. Pat. No. 7,448,995.

Thus, the device 1 may be inserted through a cannula and the tip 4 of the device 1 then steered toward the target tissue, for example, the tip 4 may be deflected a predetermined number of degrees relative to the cannula, thereby allowing the tip 4 and brushes 12 to approach the target tissue approximately perpendicular to the target tissue surface.

In an exemplary embodiment the device 1 and/or the tip 4 can be sterilized using steam and pressure, e.g., by autoclaving, wherein the device 1 is now substantially free of bacteria or viral contaminants.

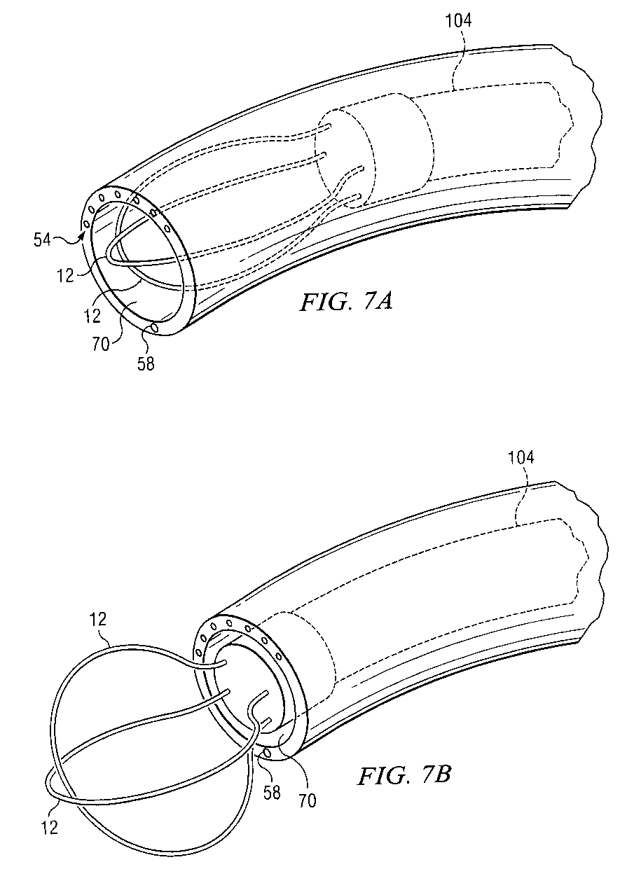

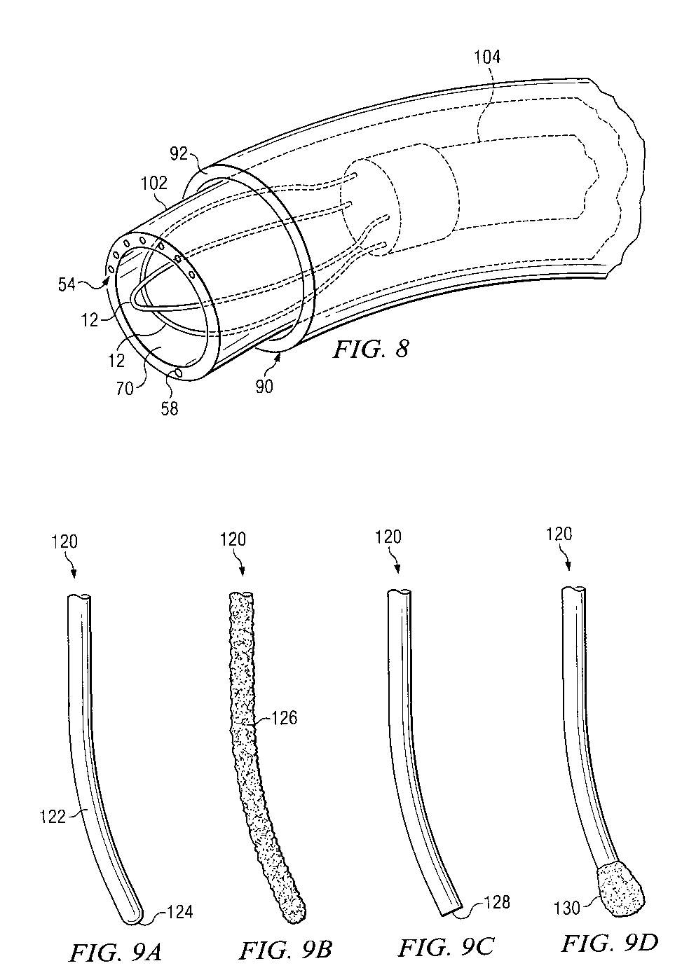

In an exemplary embodiment, the invention involves the use of a small bristles 12 extending substantially parallel to the drive shaft, having an appropriate degree of elasticity, and that expand in diameter once extended beyond the end of a channel 70 into the periodontal area to be cleaned (FIGS. 7A and 7B). Expansion of bristles 12 may be due to either centripetal force and/or a pre-formed shape. The rotating action of the bristles 12 prepares the surfaces of the root and adjacent bone by scrapping off any bacterial film, necrotic tissue, plaque, calculus, stain, cementum, and/or dentin. In addition, the bristles 12 may be used to roughen the surface of the bone. The invention may be designed to both break-up and remove dislodged material, for example, by applying a vacuum. In another exemplary embodiment, the invention may be configured to dislodge or breakup primarily soft tissue, for example, lose soft tissue as opposed to healthy soft tissue, and a minimal amount of hard tissue within the area to be treated or the disease pocket/cavity. The instrument can specifically be design to remove only unwanted tissues by use of optimal material for the bristles 12 and adjustment of their rotating speeds. The generated debris particles may be aspirated out of the defect area using a separate irrigation and/or suction device via a channel incorporated into the device (not shown). The device may also be inserted through a cannula in order to access the proper dental site. Alternatively, the device may have and extendable sheath 90 that has a leading edge 92 that is capable of being extended beyond the distal end of the main channel 70 so as to form a retractable cannula tip 92. In FIG. 8, the extendable sheath 90 has an inner diameter that is large enough to surround and slide along the outer surface 102 of the channel 70, wherein the drive shaft 104 is rotatably held in the channel 70.

In FIG. 9A-D variations on the bristles 120 are illustrated. FIG. 1A illustrates a bristle 120 that has a smooth surface 122 and a rounded tip (distal end) 124. FIG. 1B illustrates a bristle 120 that has a rough surface 126. FIG. 1C illustrates a bristle 120 that has a squared or sharp angle tip (distal end) 128. FIG. 1D illustrates a bristle 120 that has a grinding or polishing material 124 applied to the distal end. The shape of the distal end may be any desirable and appropriate shape, the surface of the bristle 120 may be smooth or rough, appropriate and desirable materials may be applied to the distal end of the bristles 120 and combinations thereof.

In another exemplary embodiment, the invention provides a method of treating a periodontal defect by inserting the tip 4 and using the bristles 12 to clean any bacterial film, plaque, necrotic tissue or other deleterious material surrounding a tooth, tooth socket, or alveolar ridge, and then applying a therapeutic agent to the area between the tooth and surrounding soft tissue. For example, a biological agent such as the GDF-5, BMP-2, BMP-4, BMP-6, BMP-7, BMP-12, TGF-β1, LMP-1, IGF-I, and/or other members of the transforming growth factor-beta (TGF-β) superfamily, can be inserted down the cannula or via a needle after the cannula has been removed.

In another exemplary embodiment, a cannula may be placed through the gum of a patient with periodontal disease via a very small hole rather than surgically cutting and reflecting back the gum tissue. A device 1 according to the invention may then be inserted through the cannula and used to prepare the site by removing bacterial film, necrotic tissue, plaque, stains, calculus, cementum and/or roughening the bone surface

The device of the present invention may be used at a low power, i.e., low rotational speed or centrifugal force, to remove plaque, soft tissue and other lower density material and the power, rotational speed or centrifugal force, may be increased in response to harder material, such as calculus.

The instrument may also have depth measurement guides on the outer surface and mechanical stops to limit its depth of penetration into the periodontal defect. Alternate mechanical preparation of the periodontal defect can be used such as an ultrasonic probe or other types of tissue ablators.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all publications cited herein are hereby incorporated by reference in their entirety.

What is claimed is:

1. A periodontal device that has the ability to loosen and/or remove any diseased or necrosed tissue in a periodontal disease pocket adjacent to a tooth with a rotating mechanical action.

2. The periodontal device of claim 1, further comprising a motor, a drive shaft rotatably connected to the motor, a plurality of bristles detachably connected to the drive shaft and a power supply casing substantially covering at least the motor and part of the drive shaft, wherein the device is configured such that the motor supplies rotational energy through the drive shaft to the plurality of bristles, and wherein the plurality of bristles have a maximal diameter of travel during rotation of less than about 8 mm.

3. The periodontal device of claim 1, wherein the drive shaft is flexible and/or steerable.

4. The periodontal device of claim 1, further comprising a first mating fitting connected to the drive shaft and a second mating fitting rotatably connected to the plurality of bristles, wherein the first and second mating fitting detachably couple the drive shaft and bristles.

5. The periodontal device of claim 1, wherein the plurality of bristles have sufficient rigidity to resist deflection due to centripetal forces during operation of the device.

6. The periodontal device of claim 1, wherein the plurality of bristles have sufficient rigidity to break-up diseased connective soft tissue and a top layer of adjacent bone surfaces to prepare a site for a regenerative therapy application.

7. The periodontal device of claim 1, further comprising a first sheath surrounding at least the drive shaft, wherein the plurality of bristles may be retracted within the first sheath.

8. The periodontal device of claim 3, further comprising an illuminating system configured to illuminate a region around the rotatable bristles, and an imaging system positioned along the length of the drive shaft such that the illuminating system illuminates tissue near the bristles and the imaging system transmits visual images of the tissue proximal to the bristles to a monitor.

9. The periodontal device of claim 8, wherein the illuminating system is a light-emitting diode or fiber optic light guide connected to a light source.

10. The periodontal device of claim 8, wherein the imaging system is a miniature video camera.

11. The periodontal device of claim 1, wherein at least one of the plurality of bristles has a rough surface.

12. The periodontal device of claim 1, wherein at least one of the plurality of bristles has a grinding material connected to the distal end of the bristle.

13. The periodontal device of claim 1, wherein the motor is an electrical motor powered by alternating current or pressurized gas.

14. The periodontal device of claim 1, wherein the motor is an electrical motor powered by a battery.

15. The periodontal device of claim 2, further comprising a housing surrounding at least the proximal ends of the bristles and the second mating fitting.

16. The periodontal device of claim 15, further comprising an extension housing surrounding at least a part of the drive shaft.

17. The periodontal device of claim 16, wherein the housing surrounding at least the proximal ends of the bristles is configured to be secured to a distal end of the extension housing.

18. The periodontal device of claim 1, further comprising a line feed mechanism in communication with the plurality of bristles.

19. The periodontal device of claim 1, further comprising a fluid irrigation and/or a suction feature for washing or lubricating the tissue and/or removing lose tissue.

20. A periodontal device that is miniaturized such that it can be used in a minimally invasive oral procedure with minimal gingival tissue disruption.
